(19) United States
(12) Patent Application Publication
(10) Pub. No.: US 2019/0022267 A1
(43) Pub. Date: Jan. 24, 2019

(54) AROMA DIFFUSER WITH TIMED HEATING FUNCTION

(71) Applicant: DONGGUAN YIH TEH ELECTRIC PRODUCTS CO., LTD., Dongguan (CN)

(72) Inventor: Ming Jen HSIAO, Miaoli County (TW)

(21) Appl. No.: 15/884,346

(22) Filed: Jan. 30, 2018

Related U.S. Application Data
(63) Continuation-in-part of application No. 15/653,362, filed on Jul. 18, 2017.
Application No. 15/653,362 is a continuation-in-part of application No. 15/862,356, filed on Jan. 4, 2018.

(51) Int. Cl. A61L 9/03

(52) U.S. Cl. CPC A61L 9/03; A61L 2209/133; A61L 2209/12; A61L 2209/11

(57) ABSTRACT

An aroma diffuser with timed heating function includes a housing, a heat insulation tank mounted in the housing, a heat-transfer unit mounted in the tank heat insulation tank, a heater disposed in contact with the bottom side of the heat-transfer unit for heating a disposable aroma capsule, and a control circuit assembly mounted inside the housing and electrically coupled with the heater. The control circuit assembly includes a timing circuit adapted for cutting off power supply from the heater after a predetermined heating time is up and simultaneously turns on one light source to give a visual indication signal to the outside of the housing through an associated light guide column, indicating that the predetermined heating time of said heater is up. The timing circuit is also capable of memorizing the progressive total time length of multiple heating operations of the heater.

### CROSS-REFERENCES TO RELATED APPLICATION

The present invention is a continuation-in-part of patent application Ser. No. 15/653,362 filed on Jul. 18, 2017 and Ser. No. 15/862,356 filed on Jan. 4, 2018.

### BACKGROUND OF THE INVENTION

### 1. Field of the Invention

The present invention relates to scent releasing devices and more specifically, to aroma diffuser with timed heating function.

### 2. Description of the Related Art

Conventional aroma diffusers do not provide any function to notice the user of the time in which the heater heated the aroma capsule. According to conventional aroma diffuser designs, the user cannot know whether the aromatic wax or essential oil contained in the applied aroma capsule has been exhausted. Because the contained aromatic wax or essential oil is sealed in the aroma capsule by a breathing film, the user cannot see through the breathing film to visually check the amount of the aromatic wax or essential oil in the aroma capsule. These are some of the issues with the use of an aroma diffuser to heat an aroma capsule. U.S. Pat. No. 8,066,420, U.S. Pat. No. 8,262,277, U.S. Pat. No. 8,147,116 disclose similar aroma diffusing night lamp system designs. However, the arrangement of the power supply, heat source and essential oil or fluid aromatic substance is not safety. In the use of aforesaid prior art designs, it is inconvenient to refurnish the aromatic substance. When in use, the user needs to take the aromatic substance out of the heat-transfer container carefully, and then to put the aromatic substance in the top accommodation open chamber of the aroma diffuser with timed heating function, avoiding direct contact of the hands with the aromatic substance. Further, after the aromatic substance is used up, the user needs to clean the heat-transfer container. Further, in the application of these prior art aroma diffuser designs, heat source can be transferred to the electronic components or the housing, causing component or housing damage.

Therefore, it is desirable to provide an aroma-diffuser that eliminates the drawbacks of the aforesaid prior art designs.

### SUMMARY OF THE INVENTION

The present invention has been accomplished under the circumstances in view. It is therefore an object of the present invention to provide an aroma diffuser, which provides a heat source isolation function to concentrate the heat source for heating an aroma capsule efficiently and can also effectively protect the electronic components of the control circuit assembly against thermal damage.

It is another object of the present invention to provide an aroma diffuser, which provides a timed heating function.

It is still another object of the present invention to provide an aroma diffuser, which uses a disposable aroma capsule for heating by a heater to release an aromatic vapor that facilitates quick replacement and avoids overflow of the fluid aromatic substance contained in the aroma capsule.

To achieve these and other objects of the present invention, an aroma diffuser with timed heating function comprises a housing, a heat-transfer unit, a heater, a control circuit assembly, and a heat insulation tank. The housing is a hollow shell comprising a first opening and an opposing second opening. The heat insulation tank is mounted in the first opening of the housing, comprising a tank opening in a top side thereof. The heat-transfer unit is mounted in the tank opening of the heat insulation tank. The heater is mounted in the heat insulation tank and kept in contact with a bottom side of the heat-transfer unit. The control circuit assembly is mounted inside the housing. The heater is electrically connected to the control circuit assembly. In application, the control circuit assembly is coupled to a power supply unit for causing the heater to heat the heat-transfer unit. Further, the control circuit assembly comprises a power switch for switching on/off the heater. The heat insulation tank isolates the heater, prohibiting the emitted heat from being transferred to the electronic components of the aroma diffuser, the control circuit assembly or the housing to cause damage.

Preferably, the control circuit assembly further comprises a plurality of light sources and a plurality of light guide columns. These light sources are respectively electrically installed in the control circuit assembly. The light guide columns each have one end thereof fastened to one respective light source and an opposite end thereof extended to the outside of the housing. Thus, the light guide columns can effectively guide emitted light from the respective light sources to the outside of the housing. The control circuit assembly further comprises a timing circuit adapted for cutting off power supply from the heater after a predetermined time is up and simultaneously turns on one light source to give a visual indication signal to the outside of the housing through the associated light guide column, indicating that the predetermined heating time of the heater is up.

Preferably, the timing circuit of the control circuit assembly can memorize the time length of each heating operation of the heater and the progressive total time length. When the progressive total time length reaches a predetermined value, the timing circuit of the control circuit assembly immediately cuts off power supply from the heater. At the same time, the heating circuit gives an instruction to turn on the corresponding light source, causing the corresponding light source to give off a visual indication signal to the outside of the housing via the associating light guide column. From this visual indication signal, the user is advised that the heating time of the heater in heating the aroma capsule has reached the set value, and the interior aromatics such as scented or essential oils have been exhausted. Thus, the user can prepare to replace the exhausted aroma capsule.

Preferably, the aroma diffuser with timed heating function is combined with a disposable aroma diffuser. In application, the disposable aroma diffuser is placed on the heat-transfer unit inside the housing.

Thus, the user can place a disposable aroma diffuser on the heat-transfer unit inside the housing for heating by the heater to release an aromatic vapor. After the aroma capsule is exhausted, the user can remote the exhausted aroma capsule from the housing for discarding or recycling. The timing circuit of the control circuit assembly can be set to indicate the set heating time and to give a signal when the set heating time is up. The timing circuit of the control circuit assembly can also memorize the time length of each heating operation of the heater and the progressive total time length. When the progressive total time length reaches a predetermined value, the timing circuit immediately cuts off power supply from the heater. At the same time, the heating circuit gives an instruction to turn on the corresponding light source, causing the corresponding light source to give off a visual indication signal to the outside of the housing via the associating light guide column. Thus, the user knows when to replace the exhausted aroma capsule.

### BRIEF DESCRIPTION OF THE DRAWINGS

FIG. 1 is an exploded view of an aroma diffuser with timed heating function in accordance with the present invention.

FIG. 2 is a schematic drawing illustrating the outer appearance of the aroma diffuser with timed heating function in accordance with the present invention.

FIG. 3 is a sectional view taken along line A-A of FIG. 2.

FIG. 4 is an oblique top elevational view of the aroma diffuser with timed heating function in accordance with the present invention.

### DETAILED DESCRIPTION OF THE PREFERRED EMBODIMENT

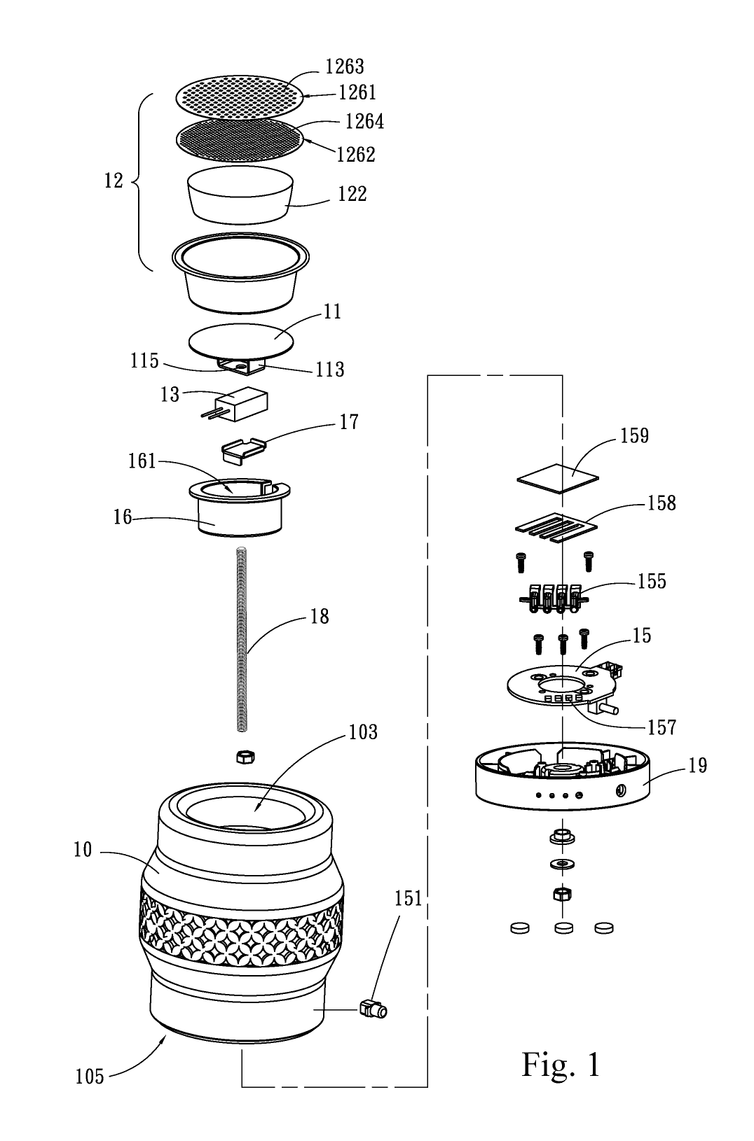

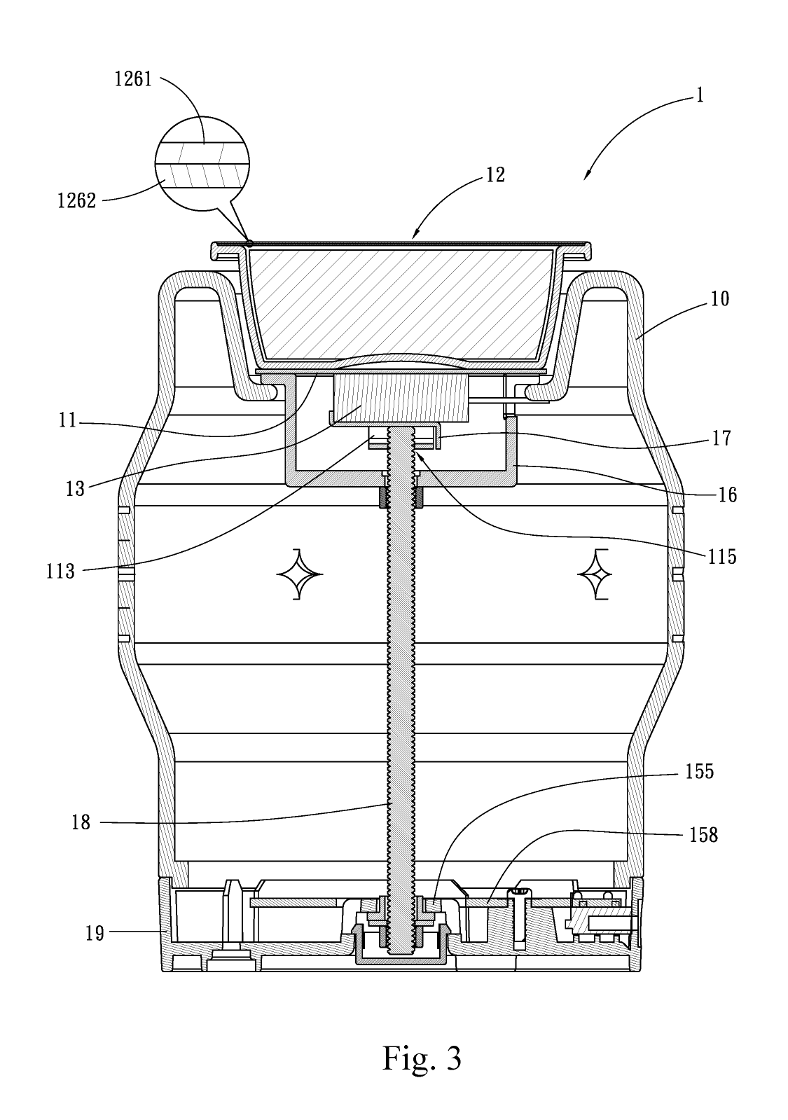

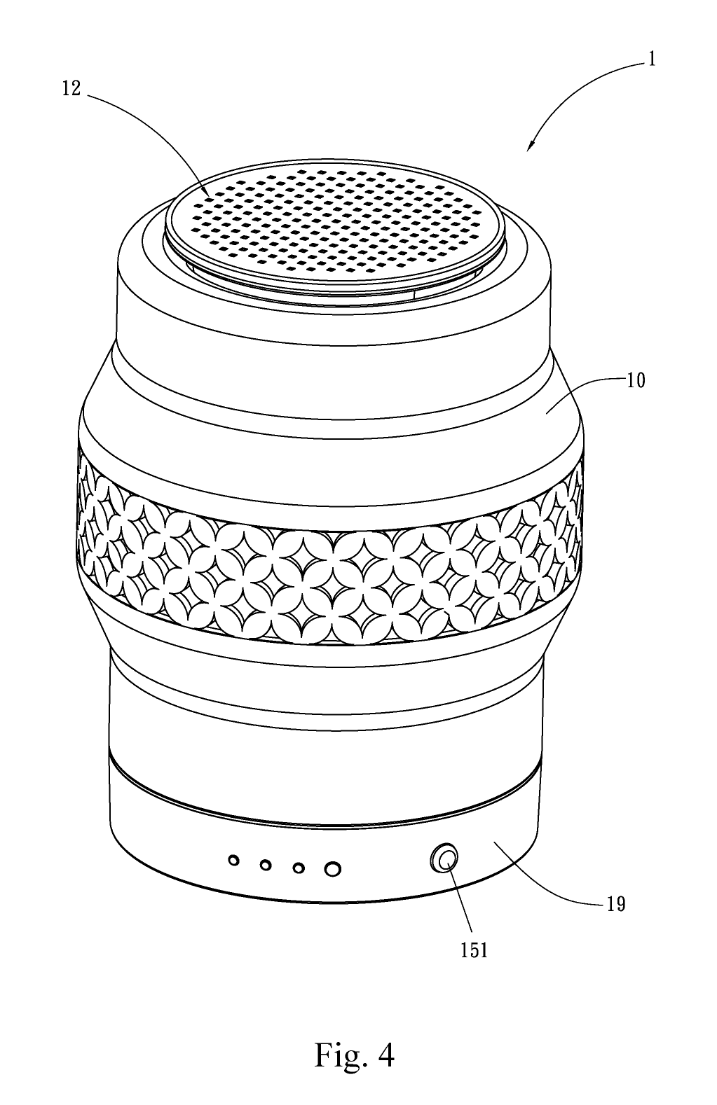

Referring to FIGS. 1-4, an aroma diffuser with timed heating function 1 in accordance with the present invention is shown. The aroma diffuser 1 comprises a housing 10, a heat-transfer unit 11, a heater 13, a control circuit assembly 15, and a heat insulation tank 16. The light-transmissive housing 10 in the present preferred embodiment is a hollow shell, comprising a first opening 103 located on one side (the top side) thereof and a second opening 105 located on an opposite side (the bottom side) thereof. The heat insulation tank 16 is mounted in the first opening 103 of the housing 10, defining a tank opening 161. The heat-transfer unit 11 is mounted in the tank opening 161 of the heat insulation tank 16. The heater 13 The heater 3 is mounted to a bottom side of the heat insulation tank 16 and kept in contact with a bottom side of the heat-transfer unit 11. The control circuit assembly 15 is mounted inside the housing 10. The heater 13 is electrically connected to the control circuit assembly 15. The control circuit assembly 15 is coupled to a power supply unit (not shown). The heater 13 is adapted for heating the heat-transfer unit 11. Further, the heater 13 can be a positive temperature coefficient (TPC) thermistor, ceramic resistor, or any other heating resistor.

The control circuit assembly 15 comprises a power switch 151 operable to switch on/off the heater 13. The heat insulation tank 16 isolate the heat generated by the heater 13, prohibiting the generated heat from being transferred to the electronic components of the aroma diffuser 1, the control circuit assembly 15 or the housing 10 to cause damage and enabling the heat generated by the heater 13 to be concentrated for heating an aroma capsule 12, and thus, the power consumption of the heater 13 can be minimized. The heat insulation tank 16 in the present preferred embodiment is shaped like a bowl with the tank opening 161 disposed in the top side. The heat insulation tank 16 can be made from temperature-resistant plastic, such as PP plastic, or heat-resistant materials, such as ceramic, carbon fiber, stone or stone powder.

Referring to FIGS. 1-4, the control circuit assembly 15 is coupled with a plurality of light sources 153 and a plurality of light guide columns 155. These light sources 153 are respectively electrically installed in the control circuit assembly 15. The light guide columns 155 each have one end thereof fastened to one respective light source 153 and an opposite end thereof extended to the outside of the housing 10. Thus, the light guide columns 155 can effectively guide emitted light from the respective light sources 153 to the outside of the housing 10. The control circuit assembly 15 comprises a timing circuit (not shown) adapted for cutting off power supply from the heater 13 after a predetermined time is up and simultaneously turning on one light source 153 to give a visual indication signal to the outside of the housing 10 through the associated light guide column 155, indicating that the predetermined heating time of the heater 13 is up. These light sources 153 can be LEDs, or small industrial or domestic lamp bulbs.

Referring to FIGS. 1-4, the timing circuit of the control circuit assembly 15 can memorize the time length of each heating operation of the heater 13 and the progressive total time length. When the progressive total time length reaches a predetermined value, the timing circuit of the control circuit assembly 15 immediately cuts off power supply from the heater 13 (the progressive total time length has reached the predetermined total time length in one heating cycle). At the same time, the timing circuit gives an instruction to turn on the corresponding light source 153, causing the corresponding light source 153 to give off a visual indication signal to the outside of the housing 10 via the associating light guide column 155. From this visual indication signal, the user is advised that the heating time of the heater 13 in heating the aroma capsule 12 has reached the set value, and the interior aromatics such as scented or essential oils have been exhausted.

Further, the light guide columns 155 can be transparent tubes or prisms made of glass, acrylic, or any other transparent plastic material.

Referring to FIGS. 1-4, when using the aroma diffuser 1, the user can put an aroma capsule 12 through the first opening 103 of the housing 10 onto the heat-transfer unit 11, and the connect the control circuit assembly 15 to a power supply unit (not shown) for causing the heater 13 to heat the heat-transfer unit 11. When the heat-transfer unit 11 is being heated, heat energy is transferred to the aroma capsule 12, causing the aroma capsule 12 to release an aromatic vapor upwardly to the outside open air. The control circuit assembly 15 can be selectively set to heat the aroma capsule 12 for one of a plurality of predetermined heating times, such as 3 hours, 6 hours or 9 hours. The setting of each predetermined heating time is linked to one respective light source 153 (such as LED). Text or symbols indicative of these predetermined heating times, such as 3 hours, 6 hours or 9 hours can be marked on the outer surface of the housing 10 adjacent to the respective light guide columns 155. When the set predetermined heating time such as 3 hours is up, the control circuit assembly 15 immediately cuts of power supply from the heater 13 and simultaneously turns on the respective light source 153, causing the associating light guide column 155 to give off a visual indication signal toward the outside of the housing 10. Through the indication of the visual indication signal of the light guide column 155 corresponding to the predetermined heating time of 3 hours, the user knows that 3 hours heating time of the heater 13 is up. At this time, the aroma diffuser 1 stops from heating the aroma capsule 12, and the control circuit assembly 15 memorizes this heating time. Thus, the user can selectively apply different heating times. When the user operates the aroma diffuser 1 to heat the aroma capsule 12 for another predetermined heating time, the control circuit assembly 15 will memorize this heating time and add this heating time to the previous record to calculate the progressive total heating time.

Referring to FIGS. 1-4, the aroma capsule 12 contains therein a certain volume of aroma wax or essential oil that can be exhausted after having been heated for a certain length of time. In the present preferred embodiment, the aroma capsule 12 will be exhausted after having been heated for totally 72 hours. After the user heated up the aroma capsule 12 for a total of 72 hours over multiple uses, for example 3 hours, 6 hours or 9 hours per each use, the control circuit assembly 15 will cuts off power supply from the heater 13, and the timing circuit will give a signal to turn on the corresponding light source 153, enabling the associated light guide column 155 to guide the emitted light to the outside of the housing 10. Through the indication of the guided light, the user knows that the progressive total heating time in which the heater 13 heated the aroma capsule 12 has reached the predetermined total heating time and the internal aromatic wax or essential oil has been exhausted, i.e., a replacement of the aroma capsule 12 is necessary.

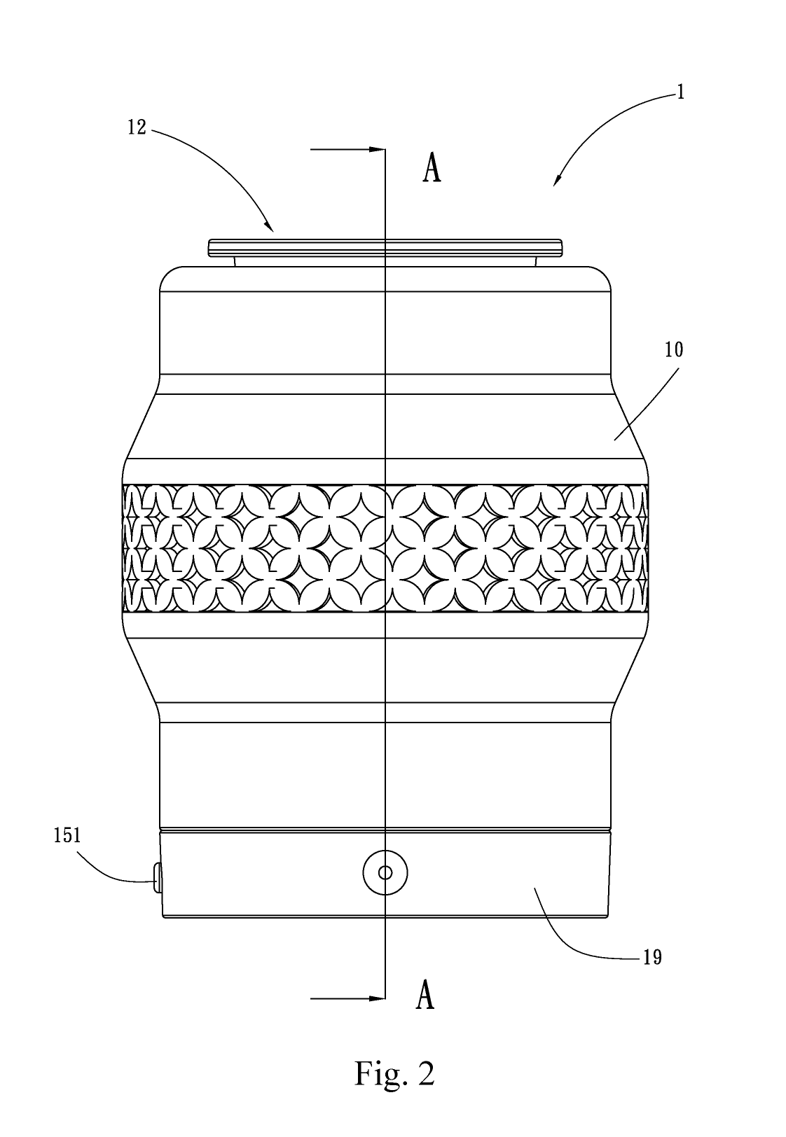

Referring to FIGS. 1 and 2, the aroma diffuser 1 further comprises a slotted light hood 158 and/or a light hood 159. The light hood 159 is preferably made of foam, cotton or fabric that is capable of absorbing or masking light. The slotted light hood 158 is adapted for masking the gap between each two adjacent light guide columns 155, prohibiting the light emitted by each light source 153 from passing through the gap between each two adjacent light guide columns 155. The light hood 159 is covered over the light guide column 155 to mask the light that is emitted by the light sources 153 and not masked by the slotted light hood 158. Thus, the light emitted by the small light sources 153 of the control circuit assembly 15 in the housing 10 can be effectively guided by the light guide columns 155 to the outside of the housing 10 for effective indication of the heating time of the aroma capsule 12.

Referring to FIGS. 2-4, the housing 10 can be selected from translucent or translucent materials, such as plastic materials, ceramic, glass, silica gel and the like. Alternatively, the housing 10 can be perforated. Further, the control circuit assembly 15 can be coupled with a decorative lamp 157 that is controllable to emit soft light for decoration. Thus, the soft light emitted by the decorative lamp 157 and the aromatic vapor released by the aroma capsule 12 to the outside of the housing 10 of the aroma diffuser 1 are combined to create an atmosphere. With the functioning of the decorative lamp 157, the aroma diffuser 1 can be used as a night lamp. Further, from the indication of the soft light emitted by the decorative lamp 157, the user is noticed that the aroma diffuser 1 under working.

Referring to FIGS. 1 and 2, the heat-transfer unit 11 further comprises a bracket 113; the heat insulation tank 16 further comprises a bottom through hole 163. The bracket 113 is located on the bottom side of the heat-transfer unit 11, comprising a mounting screw hole 115. The aroma diffuser 1 further comprises a gasket 17, a connection member 18, and a base 19. The base 19 is fastened to the second opening 105 of the housing 10. The heater 13 has one side thereof abutted against the bottom side of the heat-transfer unit 11. The gasket 17 is fastened to the heater 13. The connection member 18 has one end thereof fastened to the base 19, and an opposite end thereof inserted through the bottom through hole 163 of the heat insulation tank 16 and threaded into the mounting screw hole 115 of the bracket 113 to stop against the gasket 17 and the heater 13. In this embodiment, the connection member 18 is a screw rod that can directly fastened to the mounting screw hole 115 or the base 19 or using a screw nut.

Thus, the heat-transfer unit 11 can be firmly secured inside the housing 10 and kept in positive contact with the heater 13 for transferring heat energy efficiently.

In the preferred embodiment of the present invention, the heat-transfer unit 11 is a circular metal sheet for evenly transferring heat energy to the aroma capsule 12.

The heat-transfer unit 11 is made of a heat conducting material, such as metal, ceramic or heat conductive glass for efficiently transferring heat energy from the heater 3 to the heat-transfer unit 11.

Referring to FIGS. 1-4, the heat-activated aroma diffuser 1 is used with an aroma capsule 12. The aroma capsule 12 is placed on the heat-transfer unit 11. The heater 3 is connected to a power source and activated to generate heat. The generated heat energy is then transferred by the heat-transfer unit 11 to heat the aroma capsule 12, causing the aroma capsule 12 to release an aromatic vapor.

Referring to FIGS. 1-4, in the preferred embodiment of the present invention, the aroma capsule 12 comprises a heat-transfer container 120 defining a top opening 1200, an aromatic substance 122 held in the heat-transfer container 120, and a breathing film bonded to the heat-transfer container 120 over the top opening 1200 and the aromatic substance 122 to allow moisture vapor to be transmitted through the material.

Referring to FIGS. 1-2, in one embodiment of the present invention, the breathing film 126 comprises a fiber fixation layer 1261 and a microporous layer 1262 of excellent moisture permeability. The fiber fixation layer 1261 defines therein multiple first pores 1263. The microporous layer 1262 defines therein multiple second pores 1264. The fiber fixation layer 1261 and the microporous layer 1262 are bonded together to form the breathing film 126 with the breathable and waterproof function. The microporous layer 1262 is preferably made from thermoplastic elastomer (TPE) compound. The fiber fixation layer 1261 is preferably made from polyester. The polyester fiber fixation layer 1261 and the thermoplastic elastomer (TPE) microporous layer 1262 are bonded together to form the breathing film 126 that is breathable. The breathable aperture of the first pores 1263 is greater than the breathable aperture of the second pores 1264, facilitating outward transmittance of air. The breathing film 126 is bonded to the heat-transfer container 120 over the top opening 1200. Through the second pores 1264 of the thermoplastic elastomer (TPE) microporous layer 1262, the aroma capsule 12 provides waterproof and moisture transmissive functions, keeping the aromatic substance 122 fresh.

The thermoplastic elastomer (TPE) microporous layer 1262 and the fiber fixation layer 1261 can be bonded together using hot-press fusion or high-frequency sealing technology. Since the first pores 1263 of the fiber fixation layer 1261 are relatively lager than the second pores 1264 of the thermoplastic elastomer (TPE) microporous layer 1262, the heated aromatic substance vapor molecules can go through second pores 1264 of the thermoplastic elastomer (TPE) microporous layer 1262 toward the first pores 1263 of the fiber fixation layer 1261.

In application, the aroma capsule 12 is heated in the heat-activated aroma diffuser 1 to release a pleasant smell at a temperature below 90° C. During the heating process, the fiber fixation layer 1261 does not shrink or expand the size and can keep the thermoplastic elastomer (TPE) microporous layer 1262 in shape, and thus, the surface of the breathing film 126 can be constantly maintained smooth without wrinkles. During the heating process, aromatic substance 122 keeps releasing aromatic vapor molecules that flow smoothly through the second pores 1264 of the thermoplastic elastomer (TPE) microporous layer 1262 and the first pores 1263 of the fiber fixation layer 1261 toward the atmosphere outside the aroma capsule 12, however, the liquid phase aromatic substance is prohibited from passing through the second pores 1264, ensuring safety application of the aroma capsule 12.

Further, the aromatic substance 122 is selected from the group consisting of aromatic wax, perfume, balsam, sesame oil mixture and essential oil. In the case an aromatic wax is used for the aromatic substance 122 of the aroma capsule 12, the heat-activated aroma diffuser 1 simply needs to heat the aromatic wax to the melting point of the aromatic wax, causing the aromatic wax to release aromatic vapor molecules through the breathing film 126 toward the outside open air. In the case a sesame oil mixture or essential oil is used for the aromatic substance 122 of the aroma capsule 12, the sesame oil mixture or essential oil is heated to release aromatic vapor molecules through the breathing film 126 toward the outside open air. When compared to related existing commercial products, the breathing film 126 of the present invention will not deform during the heating process, and the released aromatic vapor molecules can flow through the breathing film 126 toward the outside open air efficiently.

Further, the heat-transfer container 120 is made from polymers. In one embodiment, the heat-transfer container 120 is made from plastics selected from the group consisting of polyester plastic, poly chloro terephthalate glycol (PCTG), polyethylene terephthalate (PET), propylthiouracil (PTU) and polypropylene (PP). The plastic heat-transfer container 120 does not melt or deform at the above-mentioned heating temperature, and can efficiently transfer heat energy from the heat-activated aroma diffuser 1 to the aromatic substance 122. In one embodiment of the present invention, the heat-transfer container 120 is made from PCTG, capable of transferring heat energy from the heat-activated aroma diffuser 1 to the aromatic substance 122 for causing the aromatic substance 122 to release a pleasant smell. Further, the heat-transfer container 120 has excellent ductility and toughness and is not easy to rapture. It will not be damaged when heating by the heat-activated aroma diffuser, and can efficiently transfer heat energy from the heat-activated aroma diffuser to the aromatic substance. When compared to fragile pottery and glass heat-transfer containers of conventional heat-activated aroma diffusers, the heat-transfer container 120 has the advantage that the top opening 1200 of the heat-transfer container 120 can easily be bonded with the breathing film 126, i.e., the thermoplastic microporous layer 1262 of the breathing film 126 can be bonded to the top opening 1200 of the heat-transfer container 120 by heat or with an adhesive. The bonding effect is better than the prior art design. After bonding, the breathing film 126 will not fall off.

Referring to FIGS. 1-4, in application, the user simply needs to put the aroma capsule 12 in the heat-activated aroma diffuser 1, and then to electrically conduct the heat-activated aroma diffuser 1, melting the aromatic substance 122 (such as aromatic wax or essential oil) to emit aromatic vapor molecules. In the heating process, the emitted aromatic vapor molecules go from the aroma capsule 12 through the second pores 1264 of the microporous layer 1262 of the breathing film 1261 and then the first pores 1263 of the fiber fixation layer 1261 to the surrounding air while the breathing film 1261 stays in shape and keeps breathing.

After the aromatic substance 122 is used up, the heat-transfer container 120 can be thrown away or recycled, and a new aroma capsule 12 can be put in the aroma diffuser with timed heating function 1. Thus, the user's hand will not be contaminated by the aromatic substance 122. Further, the user does not necessary to clean the aroma diffuser with timed heating function 1. Even if the aroma diffuser with timed heating function 1 falls accidentally during application, the melted aromatic substance 122 of the aroma capsule 12 is still held in the heat-transfer container 120 by the breathing film 126 and will not flow to the outside of the aroma diffuser with timed heating function 1 to cause contamination or dangers.

Although a particular embodiment of the present invention has been described in detail for purposes of illustration, various modifications and enhancements may be made without departing from the spirit and scope of the invention. Accordingly, the invention is not to be limited except as by the appended claims.

What the invention claimed is:

1. An aroma diffuser, comprising:
a housing in the form of a hollow shell, said housing comprising a first opening located on one side thereof and second opening located on an opposite side thereof;
a heat insulation tank mounted in said first opening of said housing, said heat insulation tank comprising a tank opening located on a top side thereof;
a heat-transfer unit mounted in said tank opening of said heat insulation tank;
a heater mounted in said heat insulation tank and disposed in contact with a bottom side of said heat-transfer unit; and
a control circuit assembly mounted inside said housing and electrically coupled with said heater and adapted for connection to a power supply unit.

2. The aroma diffuser as claimed in claim 1, wherein said control circuit assembly comprises a timing circuit.

3. The aroma diffuser as claimed in claim 2, wherein said control circuit assembly further comprising a plurality of light sources and a plurality of light guide columns, said light sources being respectively electrically installed in said control circuit assembly, said light guide columns each having one end thereof connected to one respective said light source and an opposite end thereof extended to the outside of said housing; said timing circuit of said control circuit assembly further is adapted for cutting off power supply from the heater after a predetermined heating time is up and simultaneously turns on one said light source to give a visual indication signal to the outside of the housing through the associated said light guide column, indicating that the predetermined heating time of said heater is up.

4. The aroma diffuser as claimed in claim 3, wherein said predetermined heating time is selectively 3 hours, 6 hours or 9 hours.

5. The aroma diffuser as claimed in claim 4, wherein said timing circuit of said control circuit assembly is capable of memorizing the time length of each heating operation of said heater and the progressive total time length of multiple heating operations of said heater, said progressive total time length being 72 hours.

6. The aroma diffuser as claimed in claim 3, further comprising a slotted light hood adapted for masking the gap between each two adjacent said light guide column to prohibit the light emitted by each said light source from passing through the gap between each two adjacent said light guide column, and/or a light hood covered over said light guide column to mask the light that is emitted by said light sources and passes through said slotted light hood.

7. The aroma diffuser as claimed in claim 1, wherein said heat-transfer unit comprises a bracket mounted at the said bottom side of said heat-transfer unit and comprising a mounting screw hole; said heat insulation tank further comprises a bottom through hole; said aroma diffuser further comprises a base mounted in said second opening of said housing, and a connection member, said connection member having one end thereof fastened to said base and an opposite end thereof inserted through said bottom through hole of said heat insulation tank and threaded into said mounting screw hole of said bracket to stop against said heater.

8. The aroma diffuser as claimed in claim 1, further comprising an aroma capsule placed on said heat-transfer unit inside said housing, said aroma capsule comprising a heat-transfer container defining a top opening, an aromatic substance put in said heat-transfer container and a breathing film having a breathable function and bonded to said heat-transfer container over said top opening to hold said aromatic substance in said heat-transfer container, said breathing film comprising a fiber fixation layer defining therein a plurality of first pores and a microporous layer bonded to said fiber fixation layer in a stack and defining therein a plurality of second pores.

9. The aroma diffuser as claimed in claim 2, further comprising an aroma capsule placed on said heat-transfer unit inside said housing, said aroma capsule comprising a heat-transfer container defining a top opening, an aromatic substance put in said heat-transfer container and a breathing film having a breathable function and bonded to said heat-transfer container over said top opening to hold said aromatic substance in said heat-transfer container, said breathing film comprising a fiber fixation layer defining therein a plurality of first pores and a microporous layer bonded to said fiber fixation layer in a stack and defining therein a plurality of second pores.

10. The aroma diffuser as claimed in claim 3, further comprising an aroma capsule placed on said heat-transfer unit inside said housing, said aroma capsule comprising a heat-transfer container defining a top opening, an aromatic substance put in said heat-transfer container and a breathing film having a breathable function and bonded to said heat-transfer container over said top opening to hold said aromatic substance in said heat-transfer container, said breathing film comprising a fiber fixation layer defining therein a plurality of first pores and a microporous layer bonded to said fiber fixation layer in a stack and defining therein a plurality of second pores.

11. The aroma diffuser as claimed in claim 8, wherein said microporous layer is made from thermoplastic elastomer; said fiber fixation layer is made from polyester.

12. The aroma diffuser as claimed in claim 9, wherein said microporous layer is made from thermoplastic elastomer; said fiber fixation layer is made from polyester.

13. The aroma diffuser as claimed in claim 10, wherein said microporous layer is made from thermoplastic elastomer; said fiber fixation layer is made from polyester.

14. The aroma diffuser as claimed in claim 11, wherein said fiber fixation layer is preferably made out of polyester, said polyester being selected from the group consisting of Tetoron and polyethylene terephthalate (PET); said thermoplastic elastomer is thermoplastic polyurethane (TPU); said heat-transfer container is selected from the material group consisting of plastic, polyester plastic, poly chloro terephthalate glycol (PCTG), polyethylene terephthalate (PET), propylthiouracil (PTU) and polypropylene (PP); said thermoplastic polyurethane (TPU) of said microporous layer of said breathing film is bonded to said heat-transfer container around said top opening.

15. The aroma diffuser as claimed in claim 12, wherein said fiber fixation layer is preferably made out of polyester, said polyester being selected from the group consisting of Tetoron and polyethylene terephthalate (PET); said thermoplastic elastomer is thermoplastic polyurethane (TPU); said heat-transfer container is selected from the material group consisting of plastic, polyester plastic, poly chloro terephthalate glycol (PCTG), polyethylene terephthalate (PET), propylthiouracil (PTU) and polypropylene (PP); said thermoplastic polyurethane (TPU) of said microporous layer of said breathing film is bonded to said heat-transfer container around said top opening.

16. The aroma diffuser as claimed in claim 13, wherein said fiber fixation layer is preferably made out of polyester, said polyester being selected from the group consisting of Tetoron and polyethylene terephthalate (PET); said thermoplastic elastomer is thermoplastic polyurethane (TPU); said heat-transfer container is selected from the material group consisting of plastic, polyester plastic, poly chloro terephthalate glycol (PCTG), polyethylene terephthalate (PET), propylthiouracil (PTU) and polypropylene (PP); said thermoplastic polyurethane (TPU) of said microporous layer of said breathing film is bonded to said heat-transfer container around said top opening.
